(19) United States
(12) Patent Application Publication
(10) Pub. No.: US 2013/0303986 A1
(43) Pub. Date: Nov. 14, 2013

(54) Vascular Access Catheter Anchor and Access Valve

(71) Applicant: Richard V. Penalosa, JR., Neptune, NJ (US)

(72) Inventor: Richard V. Penalosa, JR., Neptune, NJ (US)

(21) Appl. No.: 13/861,772

(22) Filed: Apr. 12, 2013

Related U.S. Application Data
(60) Provisional application No. 61/646,585, filed on May 14, 2012.

(51) Int. Cl. A61M 25/00

(52) U.S. Cl. CPC A61M 25/0097
USPC 604/118

(57) ABSTRACT

Provided is a venous catheter having an anchoring system and access valve for attachment to intravenous lumen tubing. The anchor includes a sloping base plate that is attached to the skin of a user for providing stability and immobility to the tubing. The sloping base plate elevates the tubing away from the skin in order to prevent catheter-related bloodstream infections. The plate can include an extension and cover for securing the tubing into position, and a shelf that allows the plate to extend beyond the extension to prevent skin contact with the tubing. The access valve is a T-shaped adapter for connecting the tubing to a dialysis machine tubing, and includes a flushing branch that eliminates the need to disconnect the tubing from the dialysis machine when flushing the dialysis vascular access. The access valve can be attached below the extension, snapped into the extension, or molded into the extension.

### CROSS REFERENCE TO RELATED APPLICATION

This application claims the benefit of U.S. Provisional Application No. 61/646,585 filed on May 12, 2012 entitled “CP Pack.” The above identified patent application is herein incorporated by reference in its entirety to provide continuity of disclosure.

### BACKGROUND OF THE INVENTION

1. Field of the Invention

The present invention relates to a catheter. More particularly, the present invention pertains to a venous catheter having an anchoring system and access valve for attachment to arterial and venous access tubing.

Hemodialysis is a medical treatment whereby waste and excess water is removed from the blood. This process is generally handled by the kidneys, which are responsible for filtering waste, excess fluid, and toxins. For patients with limited kidney function or with renal failure, toxins may build up in the body, blood pressure can rise, and excess fluid can collect in the body's tissues, leading to edema. For patients suffering from partial or total loss of kidney function, kidney dialysis is often required. Kidney dialysis is a form of treatment that utilizes a dialysis machine to filter toxins, wastes, salt, and excess fluid from the blood.

One of the most common types of dialysis is called hemodialysis, whereby a patient's blood is pumped through a dialyzer for filtration. For this type of dialysis, an entrance into one of the patient's blood vessels is required so that the patient can be easily connected to the dialyzer for treatment. The entrance that is created is called the vascular access, and is the location on the body where blood is removed and then returned. This can be done with a venous catheter, whereby a thin plastic tube is inserted into a vein in the neck or groin. The problem with venous catheters is that they are held in position with an apparatus that allows the catheter to make direct contact with the skin where it can collect bacteria, thereby creating a high potential for catheter-related bloodstream infections. This is caused by the presence of bacteraemia originating from the skin surrounding the catheter, and is one of the most frequent, lethal and costly complications related to catheterization.

The present invention overcomes the problems inherent in venous catheterization by providing an apparatus that reduces the risk of catheter-related bloodstream infections. The device comprises a venous catheter having an anchoring system and access valve for attachment to lumin tubing. The anchoring system includes a sloping plate with a top surface having a pair of provisions for the arterial and venous access tubing, and a bottom surface that adheres to the skin of the patient. The sloping plate keeps the top surface elevated as it moves towards the opposite end, thereby preventing the catheter access tip from making contact with the skin. In addition, the device prevents the catheter from being accidently pulled or disconnected from the dialysis machine. The access valve comprises a T-shaped connector tube to attach the dialysis vascular access tip with the dialysis machine tubing. The access valve prevents the dialysis vascular access from requiring disconnection from the dialysis machine, which lowers the risk of blood loss and contamination.

2. Description of the Prior Art

Devices have been disclosed in the prior art that relate to anchoring systems and access valves for a venous catheter. These include devices that have been patented and published in patent application publications. These devices generally relate to catheter anchors. The following is a list of devices deemed most relevant to the present disclosure, which are herein described for the purposes of highlighting and differentiating the unique aspects of the present invention, and further highlighting the drawbacks existing in the prior art.

Specifically, U.S. Pat. No. 8,105,289 to Bierman discloses an anchoring system for securing a dialysis catheter to a patient, comprising an anchor pad and a retainer. The anchor pad is attached to the skin of the patient by an adhesive layer on one side of the pad. The retainer is disposed upon the surface of the anchor pad opposite the adhesive layer, and includes a base, cover, and adhesive spot. A groove upon the base is arranged to receive a Y-site portion of a dialysis catheter where the lumens merge distal to the insertion site. A post also protrudes from the base to the cover at a position which is disposed between the two distal branches of the dialysis catheter. The cover closes over the base, securing the Y-site between the groove and the post. The contact between the retainer, adhesive spot and catheter inhibits inadvertent motion of the catheter upon the patient. The Bierman device differs in purpose and structure from the present invention. The Bierman device is meant to stabilize the Y-site base, whereas the present invention stabilizes the arterial and venous tubing.

In addition to the '289 device, U.S. Pat. No. 8,025,643 to Bierman discloses an anchoring system that secures a catheter to the body of a patient and prevents axial movement of the catheter without meaningfully impairing fluid flow. The device comprises an anchor pad that adheres to the patient's skin and supports a retainer that includes a keeper and latch that moves the retainer between an open and a closed position. The retainer further includes one or more retention mechanisms that inhibit axial movement of the catheter relative to the retainer when the catheter is secured therein. The '643 device also stabilizes the Y-site base of the dialysis vascular catheter, and does not elevate the arterial and venous tubing to lessen the chance of potential contamination.

U.S. Pat. No. 5,807,356 to Lawrence discloses a catheter with a valve for implantation in a vascular structure. The catheter is in the general shape of a T with the top of the T implanted within the lumen of a vascular structure, and the leg of the T extending out of the vascular structure through an incision in the vascular structure. The lumen of the implanted portion of the catheter completely occupies the lumen of the vascular structure, causing all blood flow through the vascular structure to be directed through the implanted portion of the catheter. A valve is placed in the wall of the implanted portion of the catheter which opens into the lumen of the T in the catheter upon application of sufficient differential pressure between the lumens of the two portions of the catheter. The '356 device is situated in the direct path of the blood flow, whereas the present invention is not in the direct path, which prevents clotting problems from occurring.

U.S. Published Patent Application No. 2008/0277926 to Inman discloses a fluid transfer assembly for transporting medicinal substances via flexible tubes and a manifold. The manifold has an inner protrusion and connector portions having inner walls. Free ends of the tubes are inserted into complementary configured inner walls of the connector portions until each of the free ends abut the inner protrusion, which creates a continuous uninterrupted passageway between the inner bores of the tubes through the manifold. While the '926 device utilizes a T-shape, it is not adapted for attachment to a dialysis vascular access tip.

Finally, Delk, U.S. Pat. No. 5,292,312 discloses a medical conduit holder for securing medical conduits to the skin of a patient. The device comprises a base plate or patch for adhesively securing to the skin, and a strap that wraps back through a slot in the strap to fully encircle the conduit or group of conduits. Pressure sensitive hook and loop type attachment surfaces are present on the upper surface of the base plate and the lower surface of the strap. The externally exposed parts of both the base patch and the strap are smooth, so as not to snag on surrounding materials. The surface of the strap that is in contact with the conduit is constructed of, or coated with, a high friction material to prevent axial slipping of the encircled conduit. The conduits may easily be oriented in any direction and may be removed or replaced without the necessity of removing the adhesive from the patient. The Delk device utilizes a hook and loop fastener to secure the conduit, and does not disclose a pair of provisions for the arterial and venous access tubing that elevate the tubing off the skin.

The devices disclosed in the prior art are apparatuses for securing medical tubing. These devices are generally intended for stabilizing the Y-site base, which anchors the dialysis vascular catheter in place. The present invention, however, does not anchor the dialysis vascular catheter itself in place, but instead stabilizes the arterial and venous tubing of the catheter. The present invention elevates the arterial and venous tubing away from the skin. The top surface of the present invention becomes more elevated as it moves away from the insertion site. This increasing elevation prevents the dialysis vascular catheter tip from making contact with potentially dirty skin, thereby lessening the chance of potential contamination. In addition, the T-shaped valve of the present invention is positioned away from the direct path of blood flow, thereby preventing potential problems with clotting, which prevents blood pooling that might result in blood clot formation.

In light of the prior art and the disclosed elements of the present invention, it is submitted that the present invention substantially diverges in design elements from the prior art. Consequently, it is clear that the present invention is not described by the art and that a need exists for an anchoring system and access valve for a venous catheter. In this regard, the instant invention substantially fulfills these needs.

### SUMMARY OF THE INVENTION

In view of the foregoing disadvantages inherent in the known types of anchoring systems and access valves for venous catheters now present in the prior art, the present invention provides a new venous catheter anchoring system and access valve wherein the same can be utilized for providing convenience for the user when preventing contamination to the vascular access tip of the catheter.

It is therefore an object of the present invention to provide a new and improved venous catheter that has all of the advantages of the prior art and none of the disadvantages.

It is another object of the present invention to provide a venous catheter that includes an anchoring system for stabilizing the extravenous tubing of the catheter assembly.

Another object of the present invention is to provide an anchoring system for a venous catheter that elevates the arterial and venous tubing away from the skin in order to reduce catheter-related bloodstream infections.

Yet another object of the present invention is to provide an access valve for a venous catheter that allows for flushing of the circuit with saline, without requiring detachment of the catheter from the arterial and venous access tubing.

Still another object of the present invention is to provide an anchoring system and access valve for a venous catheter that can be individually detached and discarded as needed.

Another object of the present invention is to provide an anchoring system and access valve for a venous catheter that utilizes a T-shaped connector tube to attach the dialysis vascular access tip with the dialysis machine tubing. The access valve prevents the dialysis vascular access from requiring disconnection from the dialysis machine, which lowers the risk of blood loss and contamination.

A final object of the present invention is to provide an anchoring system and access valve for a venous catheter that is not positioned in the direct path of blood flow, and thereby reduces formation of blood clots within the catheter lumen.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and the manner in which it may be made and used may be better understood after a review of the following description, taken in connection with the accompanying drawings wherein like numeral annotations are provided throughout.

FIG. 1 is a view of the anchor plate and access valves attached to arterial and venous tubing.

FIG. 2 is a view of the access valve connector.

FIG. 3 is an exploded view the catheter tubing anchor, access valve connectors, and dialysis tubing.

FIG. 4 is a view of an alternate embodiment of the present invention, wherein the access valves snap into the catheter tubing anchor.

FIG. 5 is a view of an alternate embodiment of the present invention, wherein the access valves are molded into the catheter tubing anchor.

FIG. 6 is a view of the catheter tubing anchor and access valves in use and attached to a venous catheter.

### DETAILED DESCRIPTION OF THE INVENTION

Reference is made herein to the attached drawings. Like reference numerals are used throughout the drawings to depict like or similar elements of the anchoring system and access valves. For the purposes of presenting a brief and clear description of the present invention, the preferred embodiment will be discussed as used for stabilizing the arterial and venous tubing of a venous catheter and for providing a means of flushing the tubing without disconnection. The figures are intended for representative purposes only and should not be considered to be limiting in any respect.

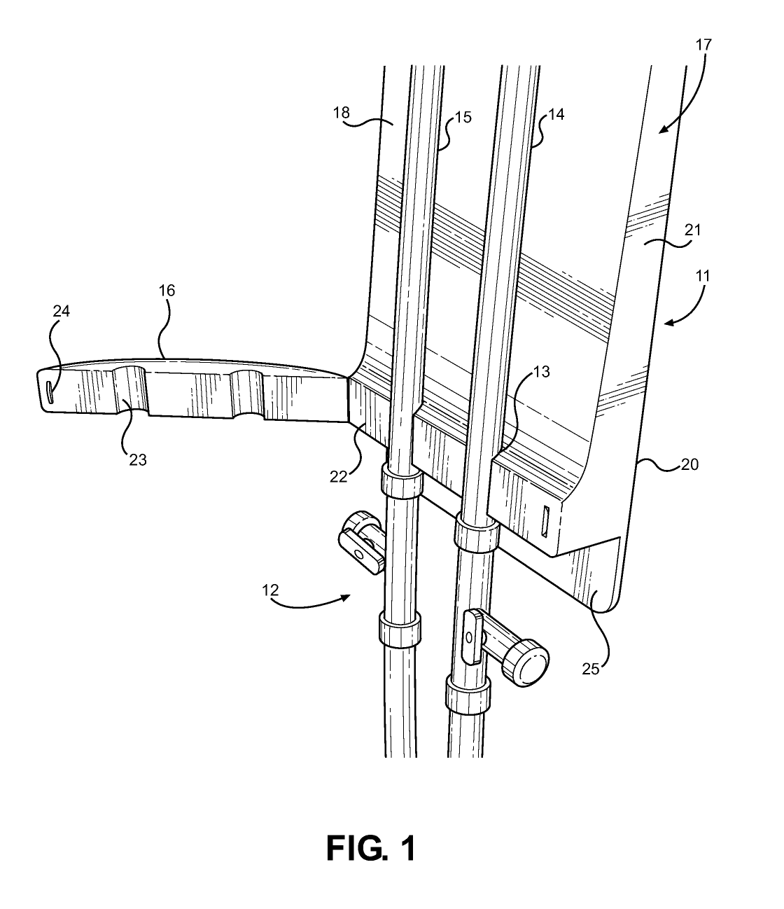

Referring now to FIG. 1, there is shown a view of the present invention, wherein the device is comprised of a venous catheter having an anchoring system and access valves for attachment to arterial and venous access tubing. The catheter tubing anchor 11 provides stability to a venous catheter that is used to access the femoral, subclavian or jugular veins, while the access valve connectors 12 provide a means of flushing the arterial 14 and venous 15 tubing of the catheter without disconnection. The anchor includes a pair of recesses 13 that secure the arterial and venous tubing of the catheter. The tubing is secured within the recess with a cover 16 that can be locked into position. This prevents accidental disconnection of the tubing either from the tubing connection of the dialysis machine or from the patient, and provides stability and immobility to the arterial and venous tubing. The device also provides elevation of the catheter from the skin, which is important during the process of connecting or disconnecting the tip of dialysis vascular access to the dialysis machine tubing, thereby preventing potential contamination with the skin. The arterial and venous tubing is secured to the anchor, and attaches to the access valve connector, for providing a means of flushing the tubing without disconnection. This affords additional safety to the patient as there is no increased risk of potential blood loss related to clamping the tubing for disconnection, and reduces the possibility of injecting air into the circuit when opened.

The anchor includes a sloping base plate 17 that attaches to the user for providing stability and immobility to the arterial and venous tubing of the catheter. The base plate is comprised of an angled face 18 for supporting the tubing, a rear adhesive face 20, and sidewalls 21. The angle of the plate elevates the arterial and venous tubing away from the skin in order to prevent catheter-related bloodstream infections. The lower end of the plate includes an extension 22 with a pair of recesses that are adapted for supporting the arterial and venous tubing. The recesses are semicircular in shape, and apply pressure to the tubing to prevent it from shifting about when secured therein. The cover is hingedly attached to the extension, and further includes a pair of semicircular recesses 23 that align with the extension recesses, thereby allowing the cover to close about the tubing and secure it in position. The distal end of the cover includes a locking means that secures the cover to the extension. The locking means is preferably a latch 24 or another suitable closure that can easily be opened and closed as desired.

The sloping base plate is constructed of a flexible material that allows the device to conform to the location on the body the plate is attached thereto. The adhesive layer includes a conventional medical-grade adhesive that is well known and available in the art. The adhesive is preferably covered with a removable film prior to use, which can be peeled off the adhesive layer for attachment to a patient. The sloping base provides elevation of the catheter from the skin, which is one of the key features of the present invention. As can be appreciated, when the tip of the arterial and venous tubing is connected or disconnected from the dialysis machine, it commonly makes contact with the skin, which can possibly cause contamination leading to catheter-related bloodstream infections. The end of the plate near the catheter access point is closer to the skin, while the end of the plate holding the tubing is elevated away from the skin, and includes a shelf 25 below the extension. The shelf allows the plate to protrude beyond the extension to prevent skin contact with the tubing, and provides access to the tip of the tubing for attachment to the dialysis machine. The distance between the extension and shelf allow a caregiver to slide his or hand therebetween to attach and detach the tubing as needed, without the tubing making contact with contaminated skin.

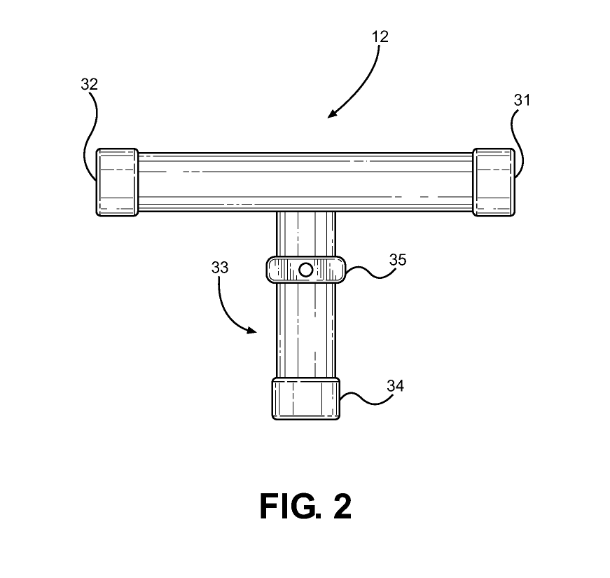

Referring now to FIG. 2, there is shown a view of the access valve connector. The connector 12 comprises a T-shaped adapter for connecting the arterial and venous tubing with the dialysis machine tubing. The valve includes a first tubing connection end 31, a second tubing connection end 32, and a flushing branch 33. The first and second connection ends are adapted for attachment to the tip of the arterial and venous access tubing at one end, and to the dialysis machine tubing at the other end. This provides an inline adapter that allows blood to flow directly therethrough. The flushing branch includes a third tubing connection end 34 and a flow control valve 35.

The access valve connector prevents the need to disconnect the dialysis vascular access from the dialysis machine tubing and expose the catheter to potential microbes and lead to contamination when flushing the dialysis vascular access. As can be appreciated, when flow through the access is restricted or stopped, the tubing must be disconnected from the dialysis machine for flushing with saline in order to detach the catheter from the wall of the vessel it is attached to. When disconnected, the tubing is exposed to air, which can lead to contamination and catheter-related bloodstream infections. In addition, when disconnection is required, the nurse or technician must properly clamp the catheter. If not properly clamped, blood loss may occur, leading to additional complications. To prevent such occurrences, a nurse or technician can attach the saline solution tubing directly to the third tubing connection end. Once the dialysis has been stopped and the saline attached, the valve can be opened, which allows the saline to flow through the tubing and into the catheter. Once flushing is complete, the valve can be closed, and the saline can be detached for continued dialysis. This not only makes the job of the nurse or technicians easier, it more importantly makes it safer for the patient, as there is no increased risk of blood loss from failing to clamp the dialysis vascular catheter access, or from injecting air into the circuit.

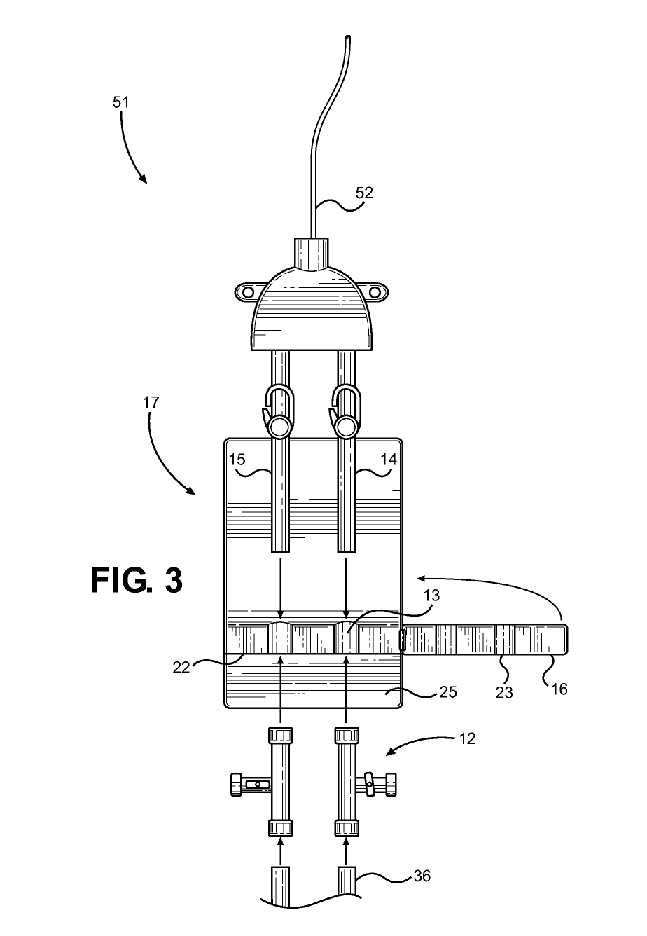

Referring now to FIG. 3, there is shown an exploded view of the catheter, catheter tubing anchor, access valve connectors, and dialysis tubing. The catheter assembly 51 includes a body 52, and arterial 14 and venous 15 access tubing extending therein. The arterial and venous access tubing attaches to the base plate 17, and is positioned with the recess 13 in the extension 22. The cover 16 locks into position so that the semicircular recesses 23 in the cover close over the top of the tubing. This prevents the tubing extending from the catheter from being pulled or kinked, which may affect the flow of therapeutic fluids, potentially leading to clotting of the dialysis filter or tubing. The tubing 36 from the dialysis machine can be directly connected to the arterial and venous tubing of the catheter in a conventional manner, or can optionally be routed through the access valve connectors 12 for use when flushing the system with saline solution. In either situation, the tips of the arterial and venous tubing extend beyond the extension and are positioned over top of the shelf 25, thereby preventing contact with contaminated skin.

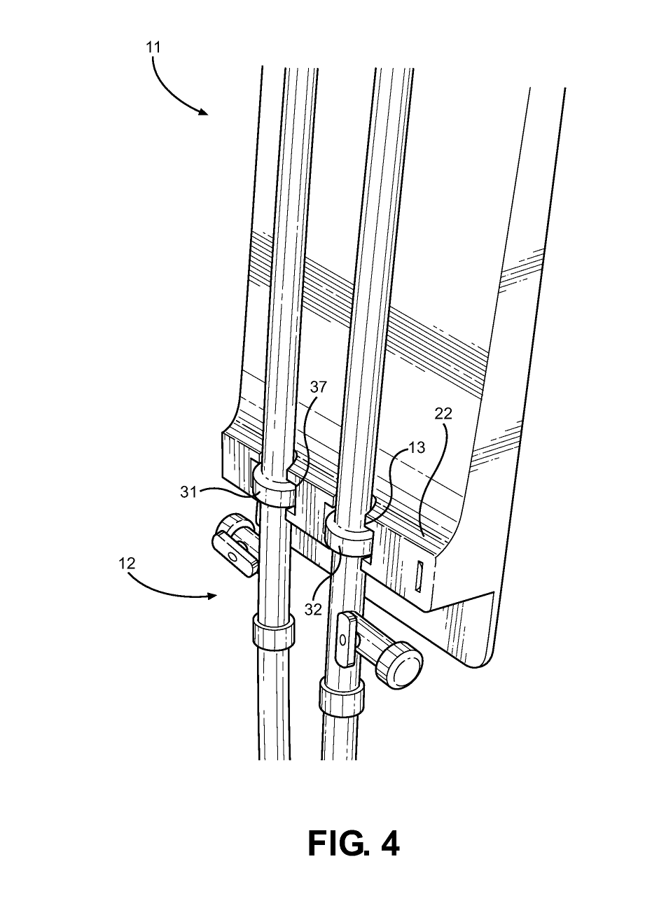

Referring now to FIG. 4, there is shown an alternate embodiment of the present invention, wherein the access valves snap into the catheter tubing anchor. In a first alternate embodiment, the extension 22 is adapted for securing the tubing connection ends 31, 32 thereto. The recesses 13 within the extension further include a pair of cutouts 37 on either side that are adapted to secure the access valve connectors 12 in place. This is ideal for situations where the valve connector will be used with the catheter tubing anchor 11. As can be appreciated, it is recommended that any material that comes in direct contact with the patient's blood be changed every 72 hours. This helps to lessen the possibility of contamination and catheter-related bloodstream infections. Similarly, materials that only contact the skin of the patient can remain in position for seven days. The ability to attach and detach the access valve connectors, which require more frequent replacement, allows for the extended use of the plate without discarding of the plate at an earlier than required time.

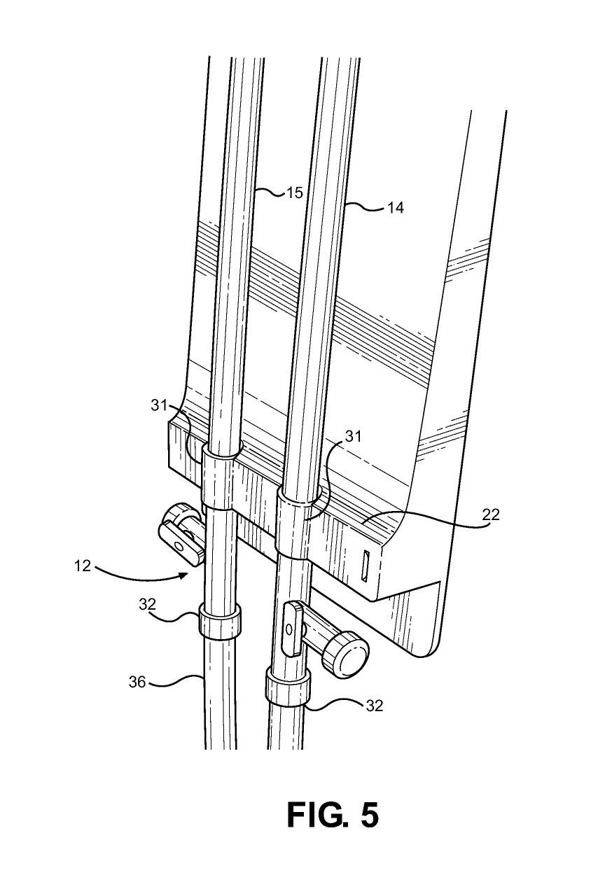

Referring now to FIG. 5, there is shown an alternate embodiment of the present invention, wherein the access valves are molded into the catheter tubing anchor. In a second alternate embodiment, the access valve connectors 12 are molded into the extension 22. In this embodiment, the arterial 14 and venous 15 tubing of the catheter attach to the first tubing connection end 31, and the tubing 36 from the dialysis machine attaches to the second tubing connection end 32. This provides a uniform structure as the access valve connectors are molded into the base plate 17. This prevents the access valve connectors from inadvertently separating from the base plate, and ensures a secure connection between the catheter and the dialysis machine.

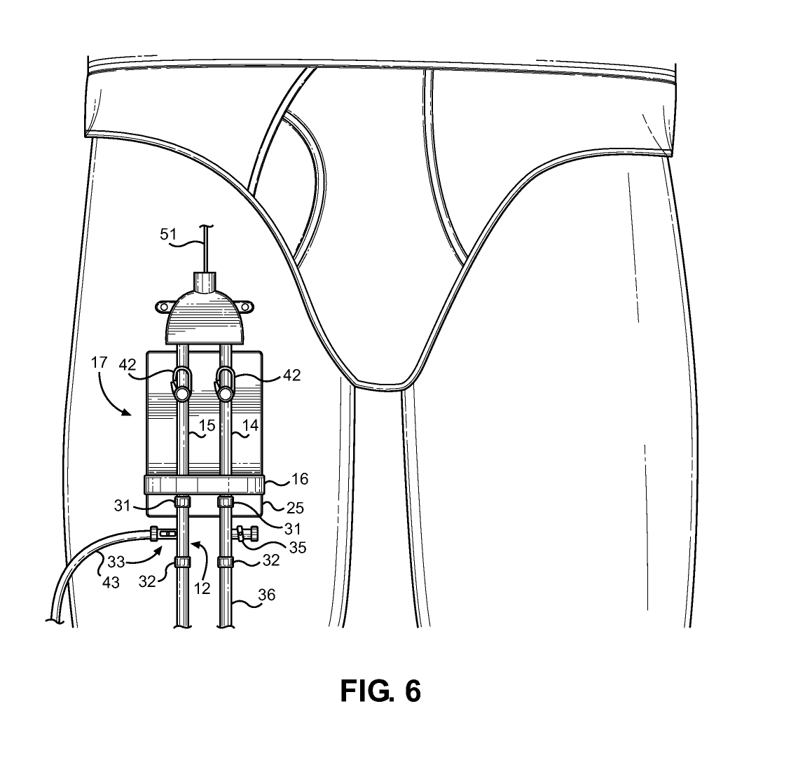

Referring now to FIG. 6, there is shown a view of the catheter tubing anchor and access valves in use and attached to a venous catheter. By way of example, the following description applies to use of the preferred embodiment plate containing a cover 16. In operation, the catheter 51 is inserted into a patient in a conventional manner, and the clamps 42 are closed to prevent blood loss. The removable film on the rear adhesive face (not shown) is peeled off, and the base plate 17 is attached to a desired location on the patient. The plate is preferably positioned so that the distal ends of the arterial 14 and venous 15 tubing extend beyond the cover and rest over the shelf 25. The tubing is then attached to the first tubing connection end 31 of the access valve connectors 12, and the cover is locked into position. The tubing 36 from the dialysis machine is attached to the second tubing connection end 32 of the access valve connectors. The flow control valve 35 is closed, and the clamps are opened. Dialysis can then be performed in a conventional manner. If flushing with saline or another solution is required, the dialysis can be stopped, and a saline tubing line 43 can be attached to the flushing branch 33. The flow control valve can be opened, allowing the saline to enter the circuit. Once flushing is complete, the flow control valve is closed, and dialysis can continue.

Overall, the present invention provides a system that lessens the possibility of catheter-related bloodstream infections. The catheter tubing anchor securely holds the catheter tubing, and prevents the catheter from being accidently pulled or disconnected from the dialysis machine. The sloping surface prevents the catheter access tip from making contact with skin, which can lead to contamination. The device can be installed in any desired location on the patient, and prevents the need for sterile gauze and Betadine, which is commonly used to create a sterile environment. The access valve allows for flushing of the circuit without disconnection, which further lowers the risk of blood loss and contamination, as the catheter tubing is not exposed to air. The components can be used together as a system, wherein the access valve is part of the anchor, or snaps into place on the anchor, or can be utilized individually. This provides a flexible system that can be customized as desired.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A venous catheter, comprising:
a body adapted for insertion into a vascular passageway, an arterial access tubing and a venous access tubing extending from said body;
a base plate that removably attaches to a user for immobilizing and stabilizing said arterial and venous tubing of said catheter; and
at least two access valve connectors having a first tubing connection end, a second tubing connection end, a flushing branch, and a flow control valve.

2) The device of claim 1, wherein said arterial and venous access tubing anchor has a sloping base plate adapted to removably secure to a patient's skin via an adhesive coating.

3) The device of claim 1, wherein said base plate further comprises:
a sloping face, a rear adhesive face, and sidewalls;
a support extension having a pair of recesses that are adapted for supporting said venous access tubing and arterial access tubing;
a cover being hingedly attached to said support extension and including a pair of semicircular recesses that align with said support extension recesses, said cover further having a locking means that secures said cover to said support extension; and
a shelf portion of said base plate extending below said support extension and is adapted to support said venous access tubing and said arterial access tubing away from a user's skin.

4) The device of claim 1, wherein said base plate further comprises:
a sloping face for supporting said arterial and venous access tubing, a rear adhesive face, and sidewalls;
a support extension having a pair of recesses, said recesses each having a cutout on either side thereof that allow said access valve connector to snap into position; and
a shelf portion of said base plate extending below said support extension, said extension is adapted to support said venous access tubing and said arterial access tubing away from a user's skin.

5) The device of claim 1, wherein said base plate further comprises:
a sloping face supporting said arterial and venous access tubing, a rear adhesive face, and sidewalls;
a support extension having at least one attached access valve connector; and
a shelf portion of said base plate extending below said support extension, said extension is adapted to support said venous access tubing and said arterial access tubing away from a user's skin

6) The device of claim 3, wherein said locking means further comprises a latch.

7) The device of claim 3, wherein said sloping base plate is constructed of a flexible material that is adapted to conform to the contour of an attachment site on a user's body.

8) The device of claim 1, wherein said access valve connector further comprises a flushing branch with a third tubing connection end.

9) The device of claim 8, wherein said access valve connector, flushing branch, and third tubing connection comprise a largely T-shaped connector.

10) The device of claim 1, wherein said access valve connector is removably connected to at least one of said arterial or venous access tubing at an end distal from said body.
